(19) United States
(12) Patent Application Publication
(10) Pub. No.: US 2003/0129640 A1
(43) Pub. Date: Jul. 10, 2003

(54) Target detection method, reagent and method for detecting SNP of DNA nucleotide sequences

(76) Inventors: Kohsuke Sasaki, Yamaguchi (JP); Tomoko Furuya, Yamaguchi (JP)

Correspondence Address: Reed Smith Hazel & Thomas LLP, Suite 1400, 3110 Fairview Park Drive, Falls Church, VA, 22042-4503, US

(21) Appl. No.: 10/326,422

(22) Filed: Dec. 23, 2002

(30) Foreign Application Priority Data
Dec. 25, 2001 (JP) 2001-392018

(51) Int. Cl. C12Q 1/68; C12M 1/34

(52) U.S. Cl. 435/6; 435/287.2

(57) ABSTRACT

A reagent and method for detecting SNPs of DNA nucleotide sequences by using a single kind of beads of the same grain size are provided. Beads on which a probe is immobilized that hybridizes with a DNA nucleotide sequence having no SNP is mixed with beads on which another probe is immobilized that hybridizes with a DNA nucleotide sequence having SNP to make a reagent. The reagent is added to a sample solution containing a fluorescence-labeled DNA nucleotide sequence under investigation and hybridized. The beads after the reaction are flown in a flow cytometer to obtain a fluorescence intensity histogram pattern, based on which SNP of the DNA nucleotide sequence under investigation is determined.

### BACKGROUND OF THE INVENTION

1. Technical Field

The present invention relates to a method of detecting a target sequence by using beads such as polystyrene beads, polypropylene beads, magnetic beads, and glass beads. The invention also relates to a reagent and method for detecting single nucleotide polymorphisms (SNPs) of DNA nucleotide sequences.

2. Background Art

A biochip (DNA microarray) is used for detecting many kinds of biopolymers, such as DNA nucleotide sequences, contained in a sample at once. The biochip takes advantage of the fact that multiple kinds of labeled targets (such as DNAs) in a sample selectively bind to multiple kinds of probes (such as DNAs) by hybridization, the probes being immobilized on a flat supporter such as a slide glass. Specifically, a target sequence in the sample is detected by means of a marker that is introduced as the target hybridizes to a spot where a probe is immobilized. By immobilizing a great number of kinds of probes on a single supporter, many kinds of targets can be detected at the same time.

Another hybridization method that has been developed uses spherical beads instead of the flat supporter. Each bead has a diameter of the order of from 100 nm to 100 μm. Probes are immobilized on the surface of the beads, so that the probes are hybridized with targets on the surface of the beads. This method, which uses the surfaces of the spheres as the site of reaction, has the advantage that the reaction efficiency and cleaning efficiency are high.

Although the method of hybridizing a probe with a target on the bead surface has a high reaction efficiency, the target that has hybridized to the probe immobilized on the beads surface must be identified by identifying the bead itself. For this reason, when there is only one kind of beads, only one kind of target can be identified. Another method is known which employs multiple kinds of beads that are made identifiable by coloring them with fluorescent pigments. But this method requires a separate laser light source for exciting the fluorescent pigments that color the beads, in addition to the laser light source for exciting the fluorescent pigments labeling the targets. As a result, the system becomes complex and expensive.

### SUMMARY OF THE INVENTION

In view of those problems of the prior art mentioned above, it is an object of the invention to provide a target detection method that employs multiple kinds of beads on which different probes are immobilized, and that enables the beads themselves to be identified in a simple manner. The invention also provides a reagent and method for detecting SNPs of a DNA nucleotide sequence by using a single kind of beads with the same grain size.

The above-mentioned object is achieved by the invention employing beads of different grain sizes that are flown in a flow cytometer to measure forward scatter. Based on the intensity of the forward scatter, the grain sizes of the beads are identified. Different probes are immobilized on the surface of the beads of different grain sizes. After hybridizing the probes with a fluorescence-labeled target, the fluorescence intensity of the beads are measured by the flow cytometer to detect the target.

The above object is also achieved as follows. Beads on which a probe is immobilized that hybridizes with a DNA nucleotide sequence having no SNP is mixed with-beads on which another probe is immobilized that hybridizes with a DNA nucleotide sequence having SNP to make a reagent. The reagent is added to a sample solution containing a fluorescence-labeled DNA nucleotide sequence under investigation and a hybridization reaction is effected. The beads after the reaction are flown in a flow cytometer to obtain a fluorescence intensity histogram pattern, based on which SNP of the DNA nucleotide sequences under investigation is determined.

In one aspect, the invention provides a target detection method comprising the steps of:

effecting a hybridization reaction between a target and a probe by mixing, in a sample solution containing a fluorescence-labeled target, beads of a first grain size having a first probe immobilized on the surface thereof and beads of a second grain size having a second probe which is different from the first probe immobilized on the surface thereof;

detecting, after the hybridization reaction, forward scatter and fluorescence simultaneously by flowing the beads in a flow cytometer; and

identifying the grain size of the beads based on the intensity of the forward scatter.

Beads of the same grain size may have the same probe immobilized thereon, and beads of different grain sizes may have different probes immobilized thereon.

In another aspect, the invention provides a reagent for detecting SNP in a DNA nucleotide sequence, comprising a mixture of first and second beads, wherein the first beads have a probe immobilized on the surface thereof which hybridizes with a DNA nucleotide sequence having no SNP, wherein the second beads differ from the first beads only in that the sequence of the probe immobilized on the surface of the second beads hybridizes with a DNA nucleotide sequence having SNP.

The first beads and the second beads may be mixed either at the same proportions or at different proportions.

In yet another aspect, the invention provides a reagent for detecting SNPs in a DNA nucleotide sequence, which comprises a mixture of:

first beads of a first grain size having a probe immobilized on the surface thereof, the probe hybridizing with a first DNA nucleotide sequence having no SNP;

second beads which differ from the first beads only in that the sequence of a probe immobilized on the surface of the second beads hybridizes with the first DNA nucleotide sequence having SNP;

third beads of a second grain size having a probe immobilized on the surface thereof the probe hybridizing with a second DNA nucleotide sequence having no SNP; and

fourth beads which differ from the third beads only in that the sequence of a probe immobilized on the surface of the fourth beads hybridizes with the second DNA nucleotide sequence having SNP.

The first and second beads are preferably mixed at different proportions, and the third and fourth beads are preferably mixed at different proportions.

The grain size of the beads may be in the range of from 0.1 μm to 1 mm.

In still another aspect, the invention provides a method of detecting SNP in DNA nucleotide sequences, which comprises the steps of:

effecting a hybridization reaction by mixing, in a sample solution containing a fluorescence-labeled DNA nucleotide sequence under investigation, first beads and second beads, the first beads having a probe immobilized on the surface thereof which hybridizes with a DNA nucleotide sequence having no SNP, the second beads differing from the first beads only in that the sequence of a probe immobilized on the surface of the second beads hybridizes with a DNA nucleotide sequence having SNP;

creating a fluorescence intensity histogram by flowing the beads, after the hybridization reaction, in a flow cytometer and measuring the fluorescence intensity; and

determining SNP in the DNA nucleotide sequence under investigation based on a peak pattern in the fluorescence intensity histogram.

The determination of SNP in the DNA nucleotide sequence under investigation may be based on whether the number of the peak is one or two.

The first and second beads are preferably mixed at different proportions. In this case, the SNP in the DNA nucleotide sequence under investigation may be determined based on the ratio of the two peaks with different fluorescence intensities in the fluorescence intensity histogram.

The material of the beads is not particularly limited. For example, polystyrene beads, polypropylene beads, magnetic beads, and glass beads may be used. By setting up filters adapted for different grain sizes of the beads at the discharge portion of the flow cytometer, the beads of different grain sizes can be recovered.

### BRIEF DESCRIPTION OF THE DRAWINGS

FIG. 1 shows the results of measuring the forward scatter from beads with a grain size of 4.5 μm.

FIG. 2 shows the results of measuring the forward and side scatter from beads with different grain sizes (4.5 μm, 6.0 μm and 10.0 μm).

FIGS. 3A to 3C show histograms of fluorescence intensity of the beads with different diameters after hybridization.

FIG. 4 shows the results of measuring the forward scatter from beads with grain size of 4.5 μm and beads with grain size of 6.0 μm.

FIGS. 5A and 5B show histograms of fluorescence intensity after hybridization of a PCR product with two kinds of beads.

FIGS. 6A to 6C show examples of histograms of fluorescence intensity for determining SNPs.

FIGS. 7A to 7F show other examples of histograms of fluorescence intensity for determining SNPs.

FIG. 8 schematically shows a system for recovering beads and biopolymers interacting on the surface of the beads.

### DETAILED DESCRIPTION OF PREFERRED EMBODIMENTS

The invention will be hereafter described by way of embodiments with reference made to the attached drawings.

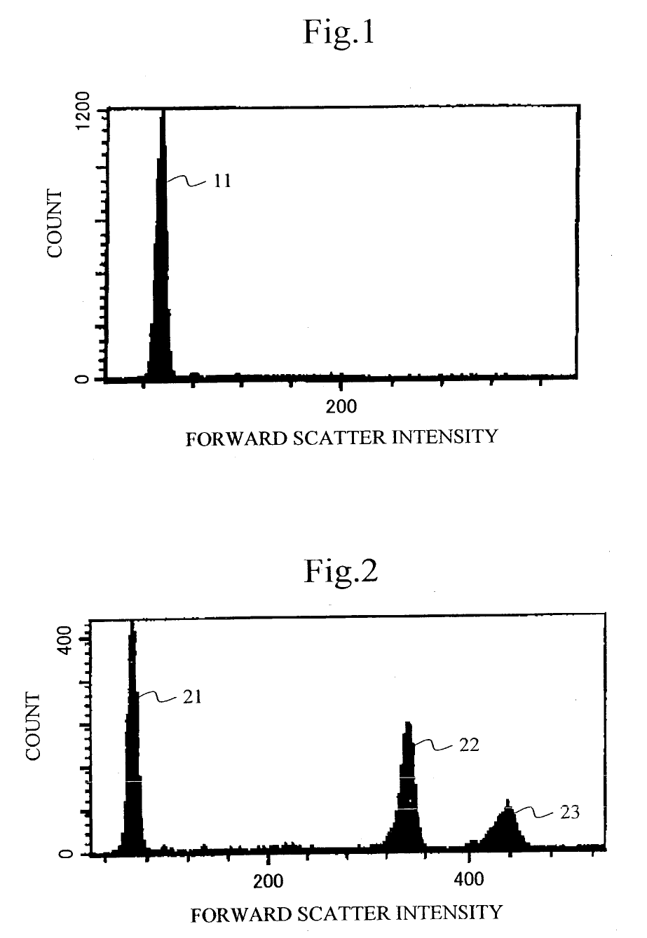

FIG. 1 is a flow cytogram showing the results of measuring the forward scatter as polystyrene beads with a grain size of 4.5 μm were flown in a flow cytometer. The horizontal axis indicates the intensity of the forward scatter (in arbitrary units), and the vertical axis indicates the number of beads counted. A steep peak 11 of counts is formed at a specific intensity of forward scatter. This shows that forward scatter of substantially the same intensity is observed when beads with the same grain size intersect the laser light of the flow cytometer. The sum of the counts in the peak 11 corresponds to the total number of the beads detected. Thus, the forward scatter intensity reflects the size (grain size) of the beads or cells, so that the grain size of beads can be identified based on the forward scatter intensity.

Various beads having a carboxyl group on the surface with grain sizes ranging from 0.05 to 90 μm are commercially available from Polysciences, Inc. or Bangs Laboratories, Inc., for example. A protein or a DNA or DNA fragment whose terminal is modified by amination can be passively adsorbed or covalently bonded to the surface of these beads. Beads with a probe such as a DNA or a protein coupled to the surface thereof are mixed in a sample solution containing a target such as a fluorescence-labeled DNA or protein, thus specifically hybridizing the target to the probe on the surface of the beads. In this way, mutations in DNA can be detected by utilizing hybridization between DNA and DNA (including RNA (ribonucleic acid), PNA (peptide nucleic acid) and LNA (locked nucleic acid), or interactions between various biopolymers, such as between protein and protein, protein and DNA, or sugars can be analyzed.

The method according to the invention will be described in detail. Three kinds of polystyrene beads with grains sizes of 4.5 μm, 6.0 μm and 10.0 μm were prepared. DNAs (probes) as shown in Table 1 below were covalently bonded to the surface of the individual beads. Specifically, amino-substituted oligonucleotides (DNAs) were covalently bonded, by means of EDC (ethylenediamine carbodiimide), to the carboxylated beads.

TABLE 1

| Type of bead | Name of the | |
|---|---|---|
| (grain size) | probe | Probe sequence |
| Polystyrene | UGT normal | TCC/TTC/GCT/CAT/TTC/AG |
| bead (4.5 μm) | UGT variant | TCC/TTC/GCG/CAT/TTC/AG |
| Polystyrene | KLK2 normal | TGC/ATT/ACC/GGA/AGT/GG |
| bead (6.0 μm) | KLK2 variant | TGC/ATT/ACT/GGA/AGT/GG |
| Polystyrene | Comt normal | TCG/CTG/GCG/TGA/AGG/AC |
| bead (10.0 μm) | Comt variant | TCG/CTG/GCA/TGA/AGG/AC |

PCR amplification was performed on 100-mer genome DNA including two SNPs detection sites. The PCR reaction condition was such that after 10 min thermal denaturation at 95° C., a cycle of PCR reaction at 94° C. for 15 sec, 55° C. for 30 sec. and 68° C. for 30 sec was repeated 35 times. DNA was fluorescence-labeled by using FITC-labeled dUTP as dNTP during PCR, and the fluorescence-labeled DNA was used for hybridization.

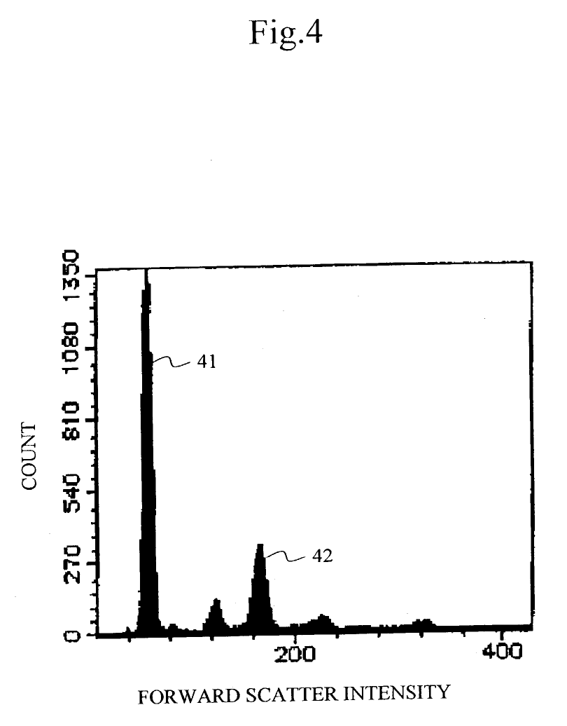

FIG. 4 shows the results of measuring forward scatter from beads of 4.5 μm grain size and beads of 6.0 μm grain size. Peak 41 with a weaker intensity indicates the forward scatter that is due to the beads of 4.5 μm grain size. Peak 42 with a stronger intensity indicates the forward scatter that is due to the beads of 6.0 μm grain size.

After hybridizing the above-mentioned PCR product (target) to the two kinds of beads of grains sizes 4.5 μm and 6.0 μm, the beads were flown in the flow cytometer to measure fluorescence intensities, based on which fluorescence intensity histograms were created. FIG. 5A shows a fluorescence intensity histogram for the beads of 4.5 μm grain size, while FIG. 5B shows a fluorescence intensity histogram for the beads of 6.0 μm grain size.

Both FIGS. 5A and 5B show two peaks in the respective fluorescence intensity histograms. This indicates that there is a mixture of those beads that emit fluorescence because of the bonding of the fluorescence-labeled PCR product thereto by hybridization, and those beads that do not emit fluorescence because of the absence of bonding of the PCR product. Namely, the fluorescence intensity histogram of FIG. 5A or 5B is providing the information that the target bonded with either the normal or variant probe and not both. If the target had bonded with both the normal and variant probes, the fluorescence marker would have been introduced into all of the beads, and only a single peak would have appeared in the fluorescence intensity histogram in the flow cytometer at the position corresponding to the above-mentioned peak with a greater intensity. Thus, in accordance with the invention, the information about SNPs can be obtained based on the number of peaks that appear in the fluorescence intensity histograms in the flow cytometer.

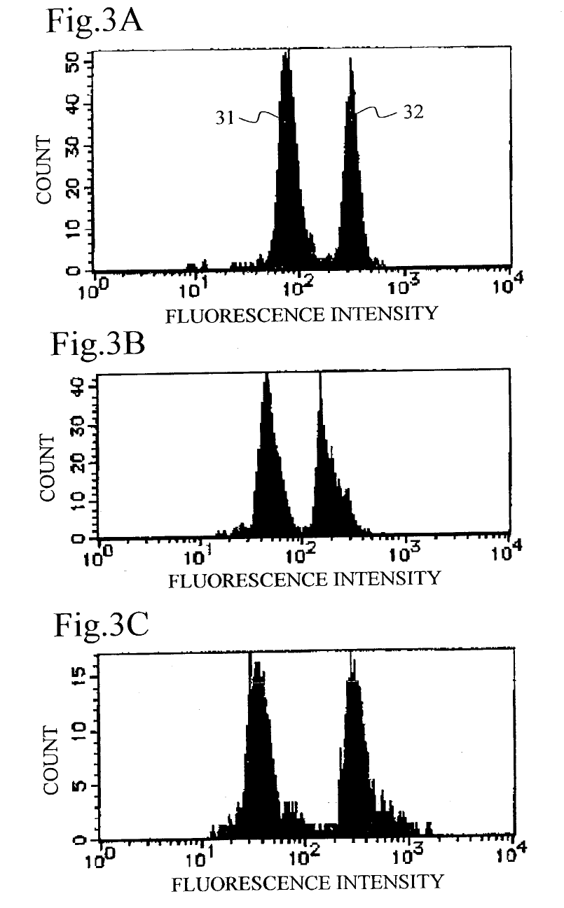

The flow cytometer can measure the forward scatter and fluorescence simultaneously for each of the beads that are successively flown. Thus, the flow cytometer stores a pair of the data about forward scatter intensity and the data about fluorescence intensity that are measured as each bead is passed through the detector in the flow cytometer. Thereafter, the data is classified according to the magnitude (corresponding to the grain size of the bead) of forward scatter intensity as shown in FIGS. 2 and 4. For each set of data with the same forward scatter intensity, a histogram, such as ones shown in FIGS. 3A, 3B, 5A and 5B is created. In this way, the information about how the target is hybridized to the different probes that are bound to the beads of the same grain size, that is the information about whether the target is of homo type or hetero type, can be obtained. By associating beads of one grain size with one gene or one DNA nucleotide sequence, SNPs of multiple genes or DNA nucleotide sequences can be simultaneously detected by using beads of multiple grain sizes.

Hereafter, a method of obtaining more detailed information, such as the kind of the substituting base, in addition to the information about whether the SNP of a DNA nucleotide sequence is of homo or hetero type, will be described. The following example employs one kind of beads of the same grain size to detect SNP of a specific gene (at a single site).

Initially, with regard to a gene of interest, beads A and beads B were prepared. Beads A had a probe covalently bonded to the surface thereof to which a nucleotide sequence having no SNPs would hybridize. Beads B had a probe covalently bonded to the surface thereof to which a nucleotide sequence of SNP would hybridize. Beads A and beads B are mixed at different proportions, such as A:B=1:2. A region of interest in a gene collected from a subject is labeled with a fluorescence marker and PCR-ampliflied to prepare a sample. This sample is added to the mixture of beads A and beads B and hybridized. Thereafter, the beads having the probe on the surface thereof to which a target in the sample hybridized are measured by a flow cytometer.

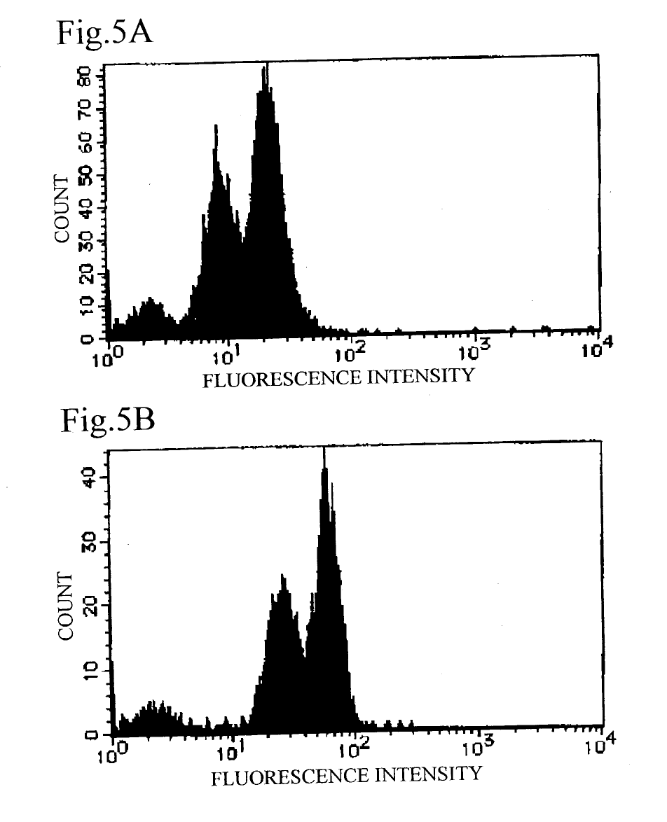

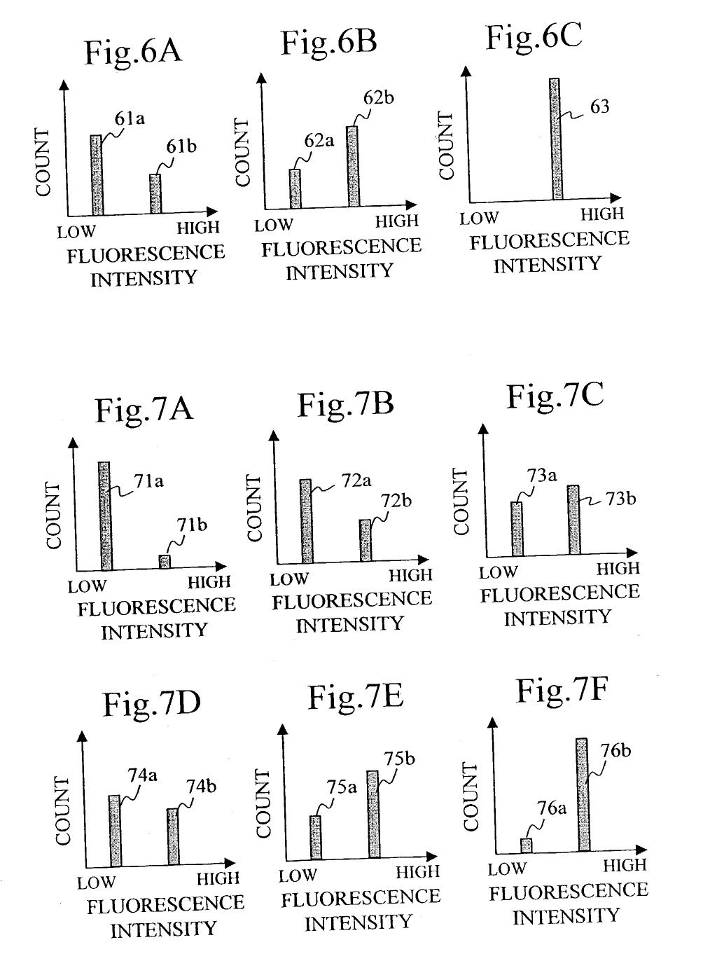

FIGS. 6A to 6C show possible three patterns of the resulting fluorescence intensity histograms. FIG. 6A shows the pattern in the case where the fluorescence marker was introduced into beads A that are fewer than beads B, namely when there is no SNPs in the gene of the subject. When the mixture ratio of beads A and beads B is such that A:B=1:2, ideally the ratio of the count for peak 61a and the count for peak 61b would be 2:1. FIG. 6B shows the pattern in the case where the fluorescence marker was introduced into beads B that are more than beads A, namely when there is SNP in the gene of the subject. In this case, ideally the ratio of the count for peak 62a and the count for peak 62b would be 1:2. FIG. 6C shows the pattern (hetero type) in the case where the fluorescence marker was introduced into both beads A and beads B, namely when the gene in the subject has both nucleotide sequences without SNPs and nucleotide sequences with SNPs. In this case, there is only one peak 63 in the fluorescence intensity histogram.

Thus, by performing the above examination and determining to which one of FIGS. 6A to 6C the pattern of the obtained fluorescence intensity histogram corresponds, SNP in the region of interest in the gene of the subject can be detected.

Next, a measuring method for the case where there are two kinds of bases that could be substituted by SNPs. In this case, with regard to the gene of interest., beads A, beads B and beads C are prepared. Beads A have a probe covalently bonded to the surface thereof to which the nucleotide sequence without SNP will hybridize. Beads B has a probe covalently bonded to the surface thereof to which the nucleotide sequence of SNP in which a base at the site of polymorphism is substituted by a first base will hybridize. Beads C has a probe covalently bonded to the surface thereof to which the nucleotide sequence of SNP will hybridize in which the base at the site of polymorphism is substituted by a second base which is different from the first base. These three kinds of beads are mixed at different proportions, such as A:B:C=1:3:5, for example. Then, a target obtained by PCR-amplifying a fluorescence-labeled region of interest in the gene collected from the subject is added to the mixture of the three kinds of beads A, B and C and hybridized. Thereafter, the beads with the probe on the surface thereof to which the target has hybridized are measured by the flow cytometer.

FIGS. 7A to 7F show possible patterns of the resultant fluorescence intensity histograms. FIG. 7A shows the case where a fluorescence marker was introduced into beads A that are fewest in number, namely when there is no SNPs in the gene of the subject. As mentioned above, when A:B:C=1:3:5, ideally the ratio of the count for peak 71a and that for peak 71b would be (3+5):1=8:1. FIG. 7B shows the case where the fluorescence marker was introduced into beads B. namely when there is SNP in the subject's gene by substitution with the first base. In this case, ideally the ratio of the count for peak 72a and that for peak 72b would be 6:3=2:1. FIG. 7C shows the pattern in the case where the fluorescence marker was introduced into beads C, namely when there is SNP in the subject's gene by substitution with the second base. In this case, ideally the ratio of the count for peak 73a and that for peak 73b would be 4:5.

FIG. 7D shows the pattern in the case where the fluorescence marker was introduced into beads A and beads B, namely when the gene of the subject contains a nucleotide sequence having no SNP and a nucleotide sequence having SNP due to substitution with the first base simultaneously. In this case, ideally the ratio of the count for peak 74a and that for peak 74b would be 5:4. FIG. 7E shows the pattern in the case where the fluorescence marker was introduced into beads A and beads C, namely when the gene of the subject contains a nucleotide sequence without SNP and a nucleotide sequence having SNP due to substitution with the second base simultaneously. In this case, ideally the ratio of the count for peak 75a and that for peak 75b would be 3:6=1:2. FIG. 7F shows the pattern in the case where the fluorescence marker was introduced into beads B and beads C, namely when the subject's gene contains a nucleotide sequence having SNP due to the first base and a nucleotide sequences having SNP due to substitution with the second base simultaneously. In this case, ideally the ratio of the count for peak 76a and that for peak 76b would be 1:8.

Thus, even when there are two kinds of bases that could possibly be substituted with SNP, the SNP in the region of interest in the subject's gene can be detected by determining to which of FIGS. 7A to 7F the fluorescence intensity histogram pattern belongs. Even when there are three or more bases that could be substituted with SNP, it can be determined whether the subject's gene has SNP or, if it does, what kind of polymorphism that is, by the same method using the fluorescence intensity histograms. Specifically, beads having multiple targets bonded to the surface thereof are mixed at different proportions. The mixture is then mixed with a target obtained by PCR-amplifying a fluorescence-labeled nucleotide sequence in a region of the gene where SNP might be present and hybridized. Then, the hybridized beads are measured by the flow cytometer.

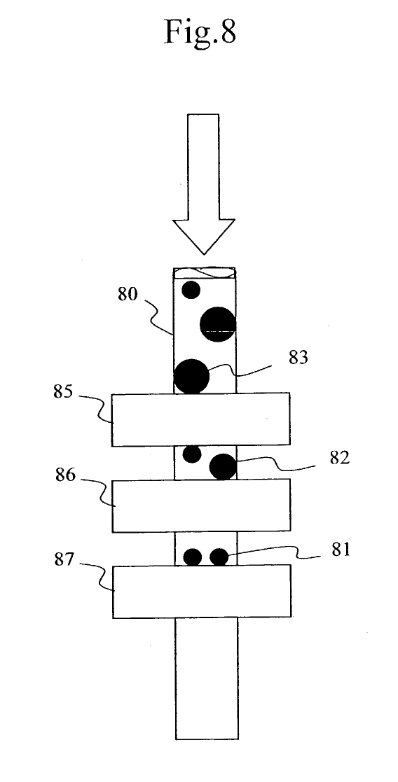

FIG. 8 schematically shows a system for recovering beads and biopolymers that are interacting on the surface of the beads after measurement in the flow cytometer. This system comprises filters 85, 86 and 87 having different meshes arranged in multiple stages in a flow path 80 of the flow cytometer. For example, when beads of three grain sizes of 4.5 μm, 6.0 μm and 10.0 μm are used, filter 85 with a mesh size of 8 μm, filter 86 with a mesh size of 5 μm, and filter 87 with a mesh size of 3 μm are arranged in this order from the upstream side. This arrangement of the filters 85 to 87 at the discharge outlet that are appropriately chosen for the grain sizes of the beads makes it possible to recover the beads, which, together with the solution, are normally discharged as waste fluid after completion of measurement in the flow cytometer. As shown, the beads of 4.5 μm grain size pass through the filters 85 and 86 but captured by the filter 87 of 3 μm mesh disposed most downstream. The beads of 6.0 μm grain size pass through the filter 85 of 8 μm mesh but captured by the filter 86 of 5 μm mesh. The beads of 10.0 μm grain size are captured by the filter 85 of 8 μm mesh disposed most upstream.

Thus, in accordance with the invention, beads can be identified by a simple method and multiple kinds of specimens can be simultaneously measured. The invention also makes it possible to determine SNPs of DNA nucleotide sequences by a simple method.

What is claimed is:

1. A target detection method comprising the steps of:
effecting a hybridization reaction between a target and a probe by mixing, in a sample solution containing a fluorescence-labeled target, beads of a first grain size having a first probe immobilized on the surface thereof and beads of a second grain size having a second probe which is different from the first probe immobilized on the surface thereof;
detecting, after the hybridization reaction, forward scatter and fluorescence simultaneously by flowing the beads in a flow cytometer; and
identifying the grain size of the beads based on the intensity of the forward scatter.

2. The target detection method according to claim 1, wherein beads of the same grain size have the same probe immobilized thereon, and beads of different grain sizes have different probes immobilized thereon.

3. A reagent for detecting SNP in a DNA nucleotide sequence, comprising a mixture of first and second beads, wherein the first beads have a probe immobilized on the surface thereof which hybridizes with a DNA nucleotide sequence having no SNP, wherein the second beads differ from the first beads only in that the sequence of the probe immobilized on the surface of the second beads hybridizes with a DNA nucleotide sequence having SNP.

4. The reagent for detecting SNP in a DNA nucleotide sequence, wherein the first beads and the second beads are mixed at the same proportions.

5. The reagent for detecting SNP in a DNA nucleotide sequence, wherein the first beads and the second beads are mixed at different proportions.

6. A reagent for detecting SNPs in a DNA nucleotide sequence, which comprises a mixture of:
first beads of a first grain size having a probe immobilized on the surface thereof, the probe hybridizing with a first DNA nucleotide sequence having no SNP;
second beads which differ from the first beads only in that the sequence of a probe immobilized on the surface of the second beads hybridizes with the first DNA nucleotide sequence having SNP;
third beads of a second grain size having a probe immobilized on the surface thereof, the probe hybridizing with a second DNA nucleotide sequence having no SNP; and
fourth beads which differ from the third beads only in that the sequence of a probe immobilized on the surface of the fourth beads hybridizes with the second DNA nucleotide sequence having SNP.

7. The reagent for detecting SNP in a DNA nucleotide sequence according to claim 6, wherein the first and second beads are mixed at different proportions, and the third and fourth beads are mixed at different proportions.

8. The reagent for detecting SNPs in a DNA nucleotide sequence according to claim 3, wherein the grain size of the beads is in the range of from 0.1 μm to 1 mm.

9. The reagent for detecting SNPs in a DNA nucleotide sequence according to claim 4, wherein the grain size of the beads is in the range of from 0.1 μm to 1 mm.

10. The reagent for detecting SNPs in a DNA nucleotide sequence according to claim 5, wherein the grain size of the beads is in the range of from 0.1 μm to 1 mm.

11. The reagent for detecting SNPs in a DNA nucleotide sequence according to claim 6, wherein the grain size of the beads is in the range of from 0.1 μm to 1 mm.

12. The reagent for detecting SNPs in a DNA nucleotide sequence according to claim 7, wherein the grain size of the beads is in the range of from 0.1 μm to 1 mm.

13. A method of detecting SNP in a DNA nucleotide sequence, which comprises the steps of:
effecting a hybridization reaction by mixing, in a sample solution containing a fluorescence-labeled DNA nucleotide sequence under investigation, first beads and second beads, the first beads having a probe immobilized on the surface thereof which hybridizes with a DNA nucleotide sequence having no SNP, the second beads differing from the first beads only in that the sequence of a probe immobilized on the surface of the second beads hybridizes with a DNA nucleotide sequence having SNP;
creating a fluorescence intensity histogram by flowing the beads, after the hybridization reaction, in a flow cytometer and measuring the fluorescence intensity; and
determining SNP in the DNA nucleotide sequence under investigation based on a peak pattern in the fluorescence intensity histogram.

14. The method of detecting SNP in a DNA nucleotide sequence according to claim 13, wherein the determination of SNP in the DNA nucleotide sequence under investigation is based on whether the number of the peak is one or two.

15. The method of detecting SNP in a DNA nucleotide sequence according to claim 13, wherein the first and second beads are mixed at different proportions.

16. The method of detecting SNP in a DNA nucleotide sequence according to claim 15, wherein SNP in the DNA nucleotide sequence under investigation is determined based on the ratio of the two peaks with different fluorescence intensities in the fluorescence intensity histogram.


SEQUENCE LISTING (10 sequences)

SEQ ID NO: 1 - DNA, 17 bp, Artificial Sequence
  Description of Artificial Sequence Synthetic DNA
tccttcgctcatttcag

SEQ ID NO: 2 - DNA, 17 bp, Artificial Sequence
  Description of Artificial Sequence Synthetic DNA
tccttcgcgcatttcag

SEQ ID NO: 3 - DNA, 17 bp, Artificial Sequence
  Description of Artificial Sequence Synthetic DNA
tgcattaccggaagtgg

SEQ ID NO: 4 - DNA, 17 bp, Artificial Sequence
  Description of Artificial Sequence Synthetic DNA
tgcattactggaagtgg

SEQ ID NO: 5 - DNA, 17 bp, Artificial Sequence
  Description of Artificial Sequence Synthetic DNA
tcgctggcgtgaaggac

SEQ ID NO: 6 - DNA, 17 bp, Artificial Sequence
  Description of Artificial Sequence Synthetic DNA
tcgctggcatgaaggac

SEQ ID NO: 7 - DNA, 24 bp, Artificial Sequence
  Description of Artificial Sequence Synthetic DNA
taaactctttccaggatggcctgc

SEQ ID NO: 8 - DNA, 24 bp, Artificial Sequence
  Description of Artificial Sequence Synthetic DNA
accatgcccccaagtgctaagaag

SEQ ID NO: 9 - DNA, 24 bp, Artificial Sequence
  Description of Artificial Sequence Synthetic DNA
atcacccagcggatggtggatttc

SEQ ID NO: 10 - DNA, 24 bp, Artificial Sequence
  Description of Artificial Sequence Synthetic DNA
tttttccaggtctgacaacgggtc